(19) United States
(12) Patent Application Publication
(10) Pub. No.: US 2026/0090683 A1
(43) Pub. Date: Apr. 2, 2026

(54) MONITORING MODULES FOR HAND HYGIENE DISPENSERS

(71) Applicant: Ecolab USA Inc., Saint Paul, MN (US)

(72) Inventors: Jeffrey Rospierski, Alden, NY (US); David L. Snodgrass, Stuart, FL (US); Andrew M. Schultz, Minneapolis, MN (US); Viktor Slobodyan, Duluth, MN (US); Cheryl A. Littau, Apple Valley, MN (US); Kenneth T. Dobizl, Mounds View, MN (US)

(21) Appl. No.: 19/263,849

(22) Filed: Jul. 9, 2025

Related U.S. Application Data
(63) Continuation of application No. 18/404,202, filed on Jan. 4, 2024, now Pat. No. 12,390,056.
Application No. 18/404,202 is a continuation of application No. 17/648,389, filed on Jan. 19, 2022, now Pat. No. 11,903,537.
Application No. 17/648,389 is a continuation of application No. 15/912,999, filed on Mar. 6, 2018, now Pat. No. 11,272,815.

(51) Int. Cl. A47K 5/12; G08B 21/24; G16H 40/20

(52) U.S. Cl. CPC A47K 5/1217; G08B 21/245; G16H 40/20

(57) ABSTRACT

A wireless dispenser beacon module for a product dispenser comprises a bottle presence trigger configured to detect one of presence or absence of a product bottle in the product dispenser; an actuation sensor configured to detect, actuation of the product dispenser; and a module controller configured to wirelessly transmit dispenser data indicative of the detected one of presence or absence of the product bottle in the product dispenser associated with each detected actuation of the product dispenser.

This application is a continuation of U.S. patent application Ser. No. 15/912,999, filed Mar. 6, 2018, which claims the benefit of U.S. Provisional Application No. 62/468,214 filed Mar. 7, 2017, both of which are incorporated by reference herein in their entirety.

### TECHNICAL FIELD

The disclosure relates to monitoring of product dispensers.

### BACKGROUND

Despite improvements in hand hygiene, stricter compliance requirements, and efforts to optimize isolation practices, hospitals and other healthcare facilities are losing the war on nosocomial or Hospital Acquired Infections (HAIs) A hospital acquired infection is an infection acquired in a hospital or other healthcare facility by a patient admitted for some reason other than that specific infection. Hospital acquired infections may include infections appearing 48 hours or more after hospital admission or within 30 days after discharge. They may also include infections due to transmission from colonized healthcare workers, or occupational exposure to infection among staff of the facility. Although the majority of hospital acquired infections are preventable, sadly their incidence has only increased.

Hospital acquired infections have become more rampant as antibiotic resistance spreads. Many factors contribute to the increased incidence of hospital acquired infections among hospital patients. For example, hospitals house large numbers of people who are sick and therefore have weakened immune systems. Medical staff move from patient to patient and see many patients a day, providing a way for pathogens to spread. Research indicates that hand hygiene practices are followed only 40% of the time by healthcare workers, even after exhaustive process improvements and training efforts. Many medical procedures, such as surgery, injections and other invasive procedures bypass the body's natural protective barriers, providing entry points for pathogens. The wide-spread use of antibiotics has contributed to the emergence of resistant strains of microorganisms in healthcare facilities and well as in the community.

Compliance with hand hygiene guidelines is considered the most effective action health care workers can take to reduce pathogen transmission in health care settings. Despite this, hand hygiene compliance remains low, and improvement efforts tend to lack sustainability.

### SUMMARY

In general, the disclosure relates to systems and associated processes that monitor product dispensers. For example, a hand hygiene compliance system may monitor, analyze and report on hand hygiene compliance at a hospital or other healthcare facility.

In one example, the disclosure is directed to a device that monitors dispense events at a hand hygiene product dispenser, comprising a bottle presence trigger configured to detect presence of a hand hygiene product bottle in the dispenser, a module controller configured to receive a dispenser actuation signal, the module controller further configured to generate dispenser data upon receipt of the dispenser actuation signal, the dispenser data including a dispense event indication and a bottle presence indication, and a wireless transceiver configured to wirelessly transmit the dispenser data upon receipt of the dispenser actuation signal.

In some examples, the module controller is configured to receive the dispenser actuation signal from a switch that detects actuation of a manual hand hygiene product dispenser. In some examples, the module controller is configured to receive the dispenser actuation signal from a switch that detects actuation of a touch free hand hygiene product dispenser. In some examples, the hand hygiene product dispenser is a manually actuated hand hygiene product dispenser. In some examples, the hand hygiene product dispenser is a touch free hand hygiene product dispenser.

In some examples, the module controller is further configured to store a dispense event count upon receipt of the dispenser actuation signal. In some examples, the bottle presence trigger comprises a switch that move s from an open position to a closed position when a product bottle is installed into the hand hygiene product dispenser; and wherein the module controller is further configured to reset a dispense event count when the switch moves from the open position to a closed position.

In some examples, the dispenser data includes the dispense event count. In some examples, the dispenser beacon module further includes an indicator that is illuminated by the module controller upon receipt of the dispenser actuation signal. In some examples, the bottle presence trigger includes one of a plunger switch, a pin switch, or a rocker switch. In some examples, the bottle presence trigger is moved to a closed position when the hand hygiene product bottle is present in the hand hygiene product dispenser.

In another example, the disclosure is directed to a dispenser beacon module that provides for wireless communication of dispenser data from a manually actuated hand hygiene product dispenser, comprising a housing having a module base and a module cover, a bottle presence trigger on an outer surface of the housing that when closed provides a bottle presence signal indicative of presence of a hand hygiene container in the hand hygiene product dispenser, a dispenser actuation switch that when closed provides a dispenser actuation signal, the module base including a slot configured to slidably receive a portion of an actuator of the manually actuated hand hygiene product dispenser, an actuation slider configured to slidably engage the portion of the actuator and close the dispenser actuation switch when the actuator is manually actuated by a user, a controller that receives the dispenser actuation signal, detects a corresponding dispense event, and stores corresponding dispense event data, wherein the controller further determines status information corresponding to the dispense event, including a battery level, a bottle presence indicator, a dispense event count, and a number of dispenses remaining, and wherein the controller wirelessly transmits the dispense event data to a remote computing device, the dispense event data including the time and date of the detected dispense event, the battery level, the bottle presence indicator, the dispense event count, and the number of dispenses remaining.

In some examples, the housing is sized to be received into a receptacle within the hand hygiene product dispenser:

In some examples, the module controller detects a change in the bottle presence trigger from closed to open to detect removal of the product container from the hand hygiene product dispenser, and detects a subsequent closure of the bottle presence trigger to detect installation of another product container into the hand hygiene product dispenser, and generates a product bottle replacement indication upon detection of the subsequent closure of the bottle presence trigger.

In some examples, the module contoller compares a number of dispenses remaining associated with the product container to a predetermined alert level to determine whether the product container was replaced before the predetermined alert level was reached.

In some examples, the module controller is further configured to communicate with an identification badge associated with a user upon detection of a dispense event and to receive user identification information from the identification badge. In some examples, the dispenser data further includes the user identification information associated with the dispense event.

In another example, the disclosure is directed to a dispenser beacon module that provides for wireless communication of dispenser data from a touch free hand hygiene product dispenser, comprising a housing having a module base and a module cover, a bottle presence trigger on an outer surface of the housing that when closed provides a bottle presence signal indicative of presence of a hand hygiene product container in the touch free hand hygiene product dispenser, a controller that receives an indication of a touch free dispenser actuation from the touch free hand hygiene product dispense, detects a corresponding dispense event, and stores corresponding dispense event data, wherein the controller further determines status information corresponding to the dispense event, including a battery level associated with the dispenser beacon module, a battery level associated with the touch free dispenser, a bottle presence indicator, a dispense event count, and a number of dispenses remaining, and wherein the controller wirelessly transmits the dispense event data to a remote computing device, the dispense event data including the time and date of the detected dispense event, the battery level associated with the dispenser beacon module, a battery level associated with the touch free dispenser, the bottle presence indicator, the dispense event count, and the number of dispenses remaining.

In some examples, the module controller detects a change in the bottle presence trigger from closed to open to detect removal of the product container from the hand hygiene product dispenser, and detects a subsequent closure of the bottle presence trigger to detect installation of another product container into the hand hygiene product dispenser, and generates a product bottle replacement indication upon detection of the subsequent closure of the bottle presence trigger.

In some examples, the module contoller compares a number of dispenses remaining associated with the product container to a predetermined alert level to determine whether the product container was replaced before the predetermined alert level was reached.

In some examples, the dispenser beacon module further includes an indicator that is illuminated by the controller upon receipt of the dispenser actuation signal. In some examples, the bottle presence trigger includes one of a plunger switch, a pin switch, or a rocker switch.

In some examples, the module controller is further configured to communicate with an identification badge associated with a user upon detection of a dispense event and to receive user identification information from the identification badge, in some examples, the dispenser data further includes the user identification information associated with the dispense event.

The details of one or more examples are set forth in the accompanying drawings and the description below. Other features and advantages will be apparent from the description and drawings, and from the claims.

### BRIEF DESCRIPTION OF DRAWINGS

FIGS. 1 and 2 show a front perspective view and a back perspective view, respectively, of an example manual dispenser beacon module.

FIGS. 3 and 4 show the internal components of an example manual dispenser beacon module with the module cover removed.

FIGS. 5A and 5B show exploded views of an example manual dispenser and example manual dispenser beacon modules.

FIG. 6 shows a perspective view of an example manual dispenser beacon module installed in a manual dispenser.

FIGS. 7 and 8 show a front perspective view and a back perspective view, respectively, of an example touch free dispenser beacon module.

FIGS. 9 and 10 show a front perspective view and a back perspective view, respectively, of the internal components of example touch free dispenser beacon module with the module cover removed.

FIGS. 11A, 11B and 12-14 show various views of portions of an example touch free dispenser with its cover removed and a touch free dispenser beacon module.

FIG. 15 is a block diagram illustrating an example implementation of the electronic components of a manual dispenser beacon module.

FIG. 16 is a block diagram illustrating an example implementation of a touch free dispenser beacon module.

FIG. 17 is a block diagram of an example hand hygiene compliance monitoring system,

FIG. 18 is a flowchart illustrating an example process by which a manual dispenser beacon module may detect manual actuations of a manual hand hygiene product dispenser and wirelessly transmit dispenser data associated with the dispense event.

FIG. 19 is a flowchart illustrating another example process by which a manual dispenser beacon module may detect manual actuations of a manual hand hygiene product dispenser and wirelessly transmit dispenser data associated with the dispense event.

FIG. 20 is a flowchart illustrating an example process by which a touch free dispenser beacon module may detect actuations of a touch free hand hygiene product dispenser and wirelessly transmit dispenser data associated with the dispense event.

FIG. 21 is a flowchart illustrating another example process by which a touch free dispenser beacon module may detect actuations of a touch free hand hygiene product dispenser and wirelessly transmit dispenser data associated with the dispense event.

### DETAILED DESCRIPTION

In general, the disclosure relates to systems and associated processes that monitor hand hygiene compliance. For example, the hand hygiene compliance system may monitor, analyze and report, on hand hygiene compliance at a hospital or other healthcare facility. The disclosure describes dispenser beacon modules that may be installed in existing hand hygiene product dispensers to provide wireless communication of hand hygiene data to or from a dispenser. In one example, a manual dispenser beacon module is configured to be used with a manually actuated hand hygiene product dispenser to monitor hand hygiene compliance events associated with the dispenser, and to wirelessly transmit hand hygiene data to or from the manual dispenser. In another example, a touch free dispenser beacon module is configured to be used with a touch free hand hygiene product dispenser to monitor hand hygiene compliance events associated with the dispenser, and to wirelessly transmit hand hygiene data to or from the touch free dispenser. Additional dispenser status information may be included in the dispenser data, such as dispenser identification information, healthcare worker identification information, current battery levels, product bottle presence/absence, number of dispenser actuations, out-of-product indications, etc.

The manual and touch free dispenser beacon modules described herein may be used with any of the systems or incorporate any of the features shown and described in U.S. Pat. No. 8,502,680 issued Aug. 6, 2013; U.S. Pat. No. 8,395,515 issued Mar. 12, 2013; U.S. Pat. No. 8,264,343 issued Sep. 11, 2012; U.S. Pat. No. 8,564,431 issued Oct. 22, 2013; U.S. Pat. No. 8,964,140 issued Mar. 18, 2014; U.S. Pat. No. 8,482,406 issued Jul. 9, 2013; U.S. Pat. No. 8,872,665 issued Oct. 28, 2014; U.S. Pat. No. 8,783,511 issued Jul. 22, 2014; and U.S. Pat. No. 8,633,816 issued Jan. 21, 2014; each of which is incorporated herein by reference in its entirety,

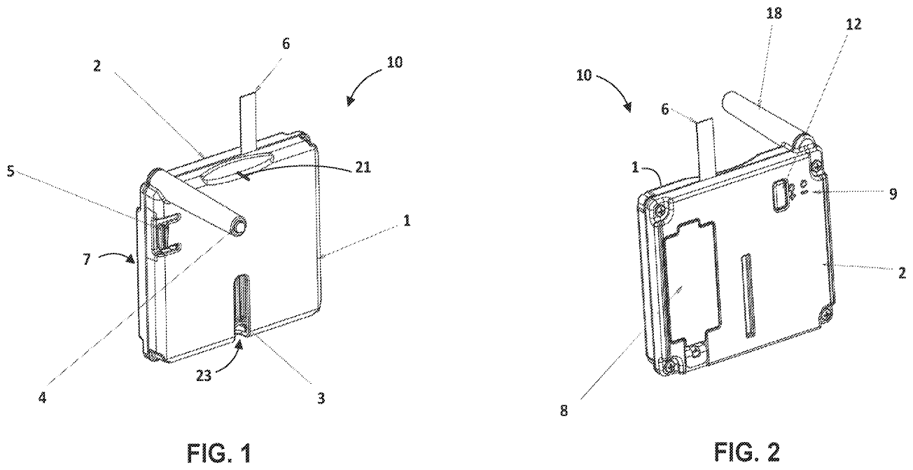

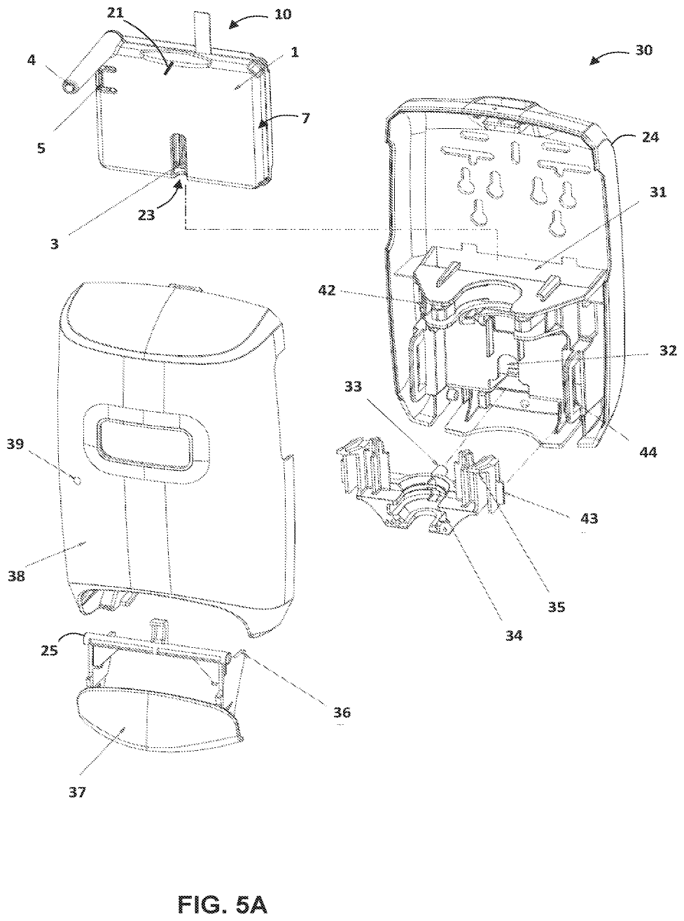

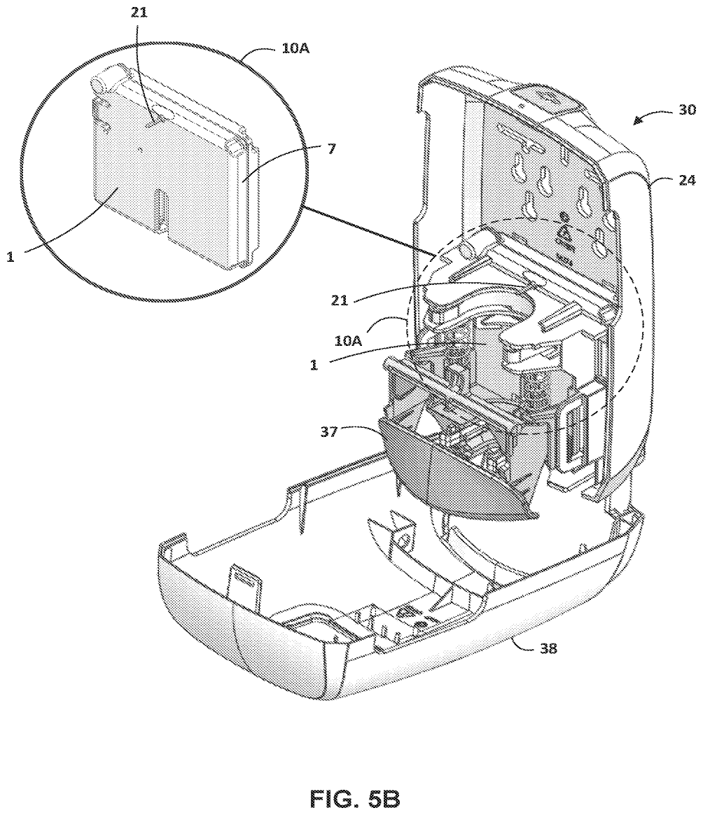

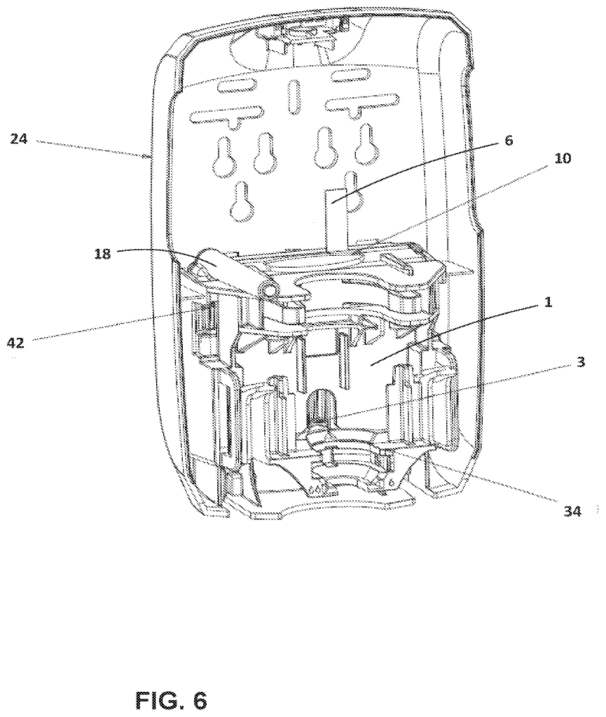

FIGS. 1 and 2 show a front perspective view and a back perspective view, respectively, of an example manual dispenser beacon module 10. Manual dispenser beacon module may be used with a manually actuated hand hygiene product dispenser to monitor hand hygiene events associated with the manual dispenser, and to wirelessly transmit hand hygiene data (including data concerning the monitored hand hygiene events) to or from the manual dispenser. Dispenser beacon module 10 includes a housing 7 having a module base 1 and a module cover 2, an actuation slider 3, an LED indicator 4, a locking mechanism 5, a release strap 6, a battery compartment door 8, and a firmware access port 12. Module base 1 is configured to form a slot 23 through which a manual dispenser actuator may engage with an actuation slider 3 (see FIG. 5A).

In some examples, the manual dispenser beacon module 10 is further configured to wirelessly transmit and/or receive communication from one or more computing device(s). For example, the beacon module 10 may receive remote software updates, remote configuration settings (e.g., range settings, product empty settings, settings for a number of dispense events before a product bottle should be refilled or replaced, etc.) from one or mare computing devices. The beacon module 10 may further communicate with one or more other beacon modules in healthcare setting, such as those associated with other dispensers, with motion detectors in a patient room or other defined area, with patient zone beacons in a patient room or other defined area, or other such devices in a healthcare setting that may be useful for monitoring of hand hygiene compliance. The beacon module 10 may be further configured to wirelessly communicate (both transmit and receive) with one or more uniquely assigned healthcare worker identification badges. For example, the beacon module 10 may be configured to communicate with a badge, obtain healthcare worker identification information from the badge, and associate a detected dispense event with the healthcare worker identification information.

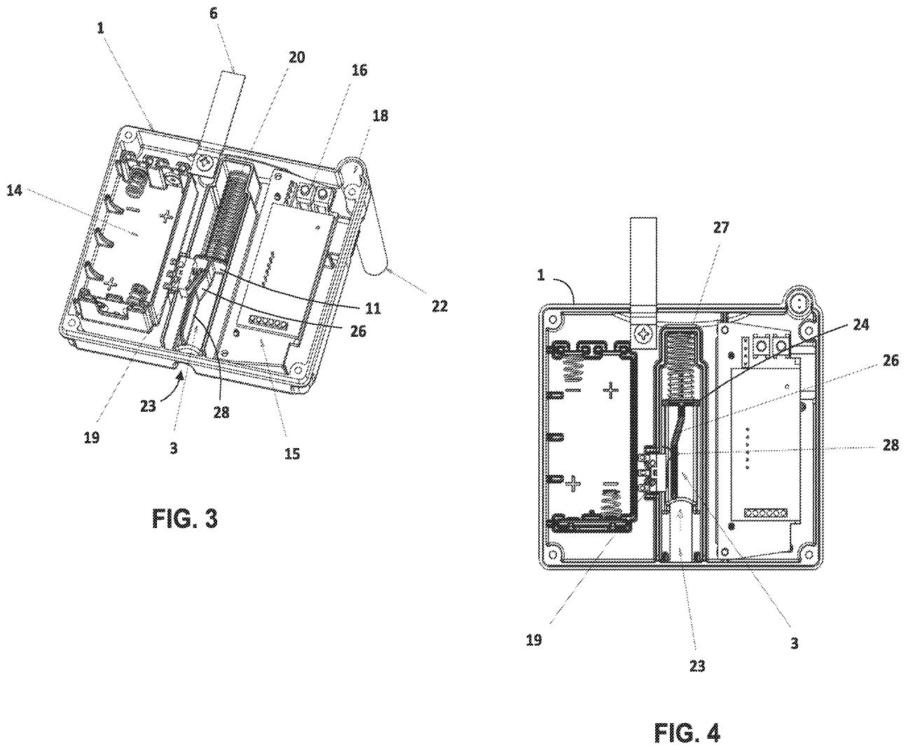

FIGS. 3 and 4 show the internal components of example manual dispenser beacon module 10 with module cover 2 removed. In FIG. 3, actuation slider 3 is in an open (at rest or non-actuated) position. In FIG. 4, actuation slider 3 is in a closed (actuated) position. The internal components of the manual dispenser beacon module 10 include a PCB assembly 15, actuation slider 3 and a return spring 20, a micro switch 19, and a battery compartment 14. In this example, battery compartment 14 is configured to receive 2 AA batteries that provide power to PCB assembly 154 in other examples, manual dispenser beacon module may be powered using different batteries or may be hard-wired to the electrical system of the building.

Pull strap 6 is fastened to module base 1 and provides for removal of module 10 from a manual dispenser (see FG 5A), PCB assembly 15 includes range adjustment buttons 16 that may be accessed through holes 9 in the module cover 2 (see FIG. 2). LED indicator 4 is connected to PCB 15 through LED tube 18 and is seated at a distal end 22 of LED tube 18. This permits LED indicator 4 to be exposed through the front cover of a manual dispenser 30 as shown in FIG. 5A. In another example, a manual dispenser beacon module IOA as shown in FIG. 5B does not include an LED light tube or LED indicator.

When the pushbar (see ref. num. 37, FIGS. 5A and 5B) is pressed by a user to dispense product, the mechanical movement of the pushbar is converted to an electrical signal by actuation slider 3 and micro switch 19, which initiates a communication sequence between the electronic components of PCB assembly 15 and other components of the beacon module. Actuation slider 3 includes a flat portion 28, a spring engagement portion 11, and a ramp portion 26 connected between the flat portion 28 and spring engagement portion 11. Switch 19 is connected and communicates with PCB assembly 15. When actuation slider 3 is at rest (FIG. 3) switch 19 is positioned with respect to a higher end of ramp portion 26 such that switch 9 is in the open position. When actuation slider 3 is moved toward the closed position (FIG. 4), ramp portion 26 of actuation slider 3 moves over switch 19 until flat portion 28 is positioned over switch 19, thus closing switch 19. This closure of switch 19 communicates to PCB assembly 15 that the dispenser has been actuated. Return spring 27 compresses as actuation slider 3 moves toward the closed position. When the dispenser bottle actuator 34 is released, return spring 27 returns actuation slider 3 to its resting position (FIG. 3).

FIG. 5A shows an exploded view of an example manual dispenser 30 and example manual dispenser beacon module 10. FIG. 5B shows a perspective view of example manual dispenser beacon module 10 installed in manual dispenser 30. Example manual dispenser 30 includes a base 24, a front cover 3S having a LED window 39, and a push bar 37. Push bar 37 snaps into dispenser cover 38. Push bar 37 freely rotates on hinge 25 once manual dispenser 30 is assembled. Manual dispenser 30 further includes a receptacle 31 configured to receive housing 7 of manual dispenser beacon module 10.

Manual dispenser beacon module 10 is configured to detect actuation of push bar 37 by a user to dispense a quantity of hand hygiene product. Manual dispenser 30 includes a bottle actuator 34 that includes slider ribs 43 that snap into mating slots 44. These features are symmetrical on both sides of dispenser base 24. Bottle actuator 34 includes a slot 32 configured to align with slot 23 of module 10 and thus allow engagement of activation slider 3 with module interface post 33 (FIG. 61). Bottle actuator 34 includes its own return springs (not shown) to return actuator 34 to its resting position.

When manual dispenser beacon module 10 is installed within manual dispenser 30, that is, when housing 7 is received within receptacle 31 of manual dispenser 30, actuation slider 3 is actuated by a module interface post 33 on bottle actuator 34. Push bar lifting ribs 36 rest against lift journals 35 on bottle actuator 34. When push bar 37 is activated by a user, lifting ribs 36 press up against lift journals 35, raising the bottle actuator 34 in slots 44, and raising module interface post 33. Interface post 33 engages the activation slider 3, lifting it to activate switch 19 and send an actuation signal to a processor on PCB assembly 15 indicating that the dispenser has been actuated.

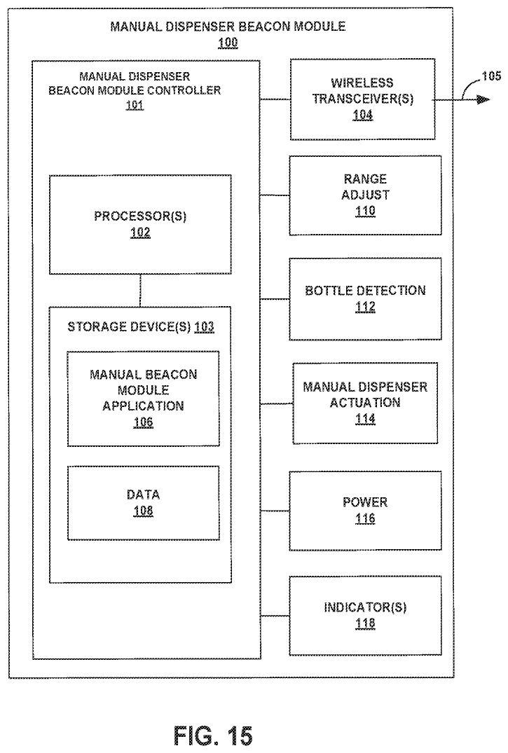

When module 10 is installed in dispenser 30 (in this example, when housing 7 is received within receptacle 31), module-side locking mechanism 5 locks module 10 to dispenser base 24 at a dispenser-side locking mechanism 42, in addition, LED indicator 4 lines up with light pipe 39 on dispenser cover 38. Indicator 4 is the visual interface with the user. A processor (see FIG. 15) on PCB assembly 15 receives the actuation signal from switch 19 and causes indicator 4 to be illuminated each time actuation of push bar 37 is detected. Once assembled, to remove module 10, locking mechanism 5 is pressed at the same time the user pulls on the release strap 6. This allows access to the batteries by removing battery door 8.

Manual dispenser beacon module 10 further includes a bottle detection switch (or bottle presence trigger) 21. Bottle presence trigger 21 is configured to be depressed or moved to the closed position when a product bottle is installed or received in the hand hygiene product dispenser. In this example, bottle presence trigger 21 is implemented using a plunger or pin switch; however, it shall be understood that any other type of switch configured to detect bottle presence could be used. When no bottle is installed in dispenser 30, bottle presence trigger 21 is not depressed (open). When a bottle of hand hygiene product is installed into manual dispenser 30, the neck of the product bottle will depress bottle presence trigger 21. When the bottle presence trigger is thus closed, switch 21 communicates a bottle present signal to the PCB assembly 15 and thus communicates to the processor on PCB assembly 15 that a bottle is installed in the dispenser. When the bottle is removed, bottle presence trigger 21 returns to its open position, communicating to PCB assembly 61 (and thus the processor thereon) that the bottle 80 has been removed. Bottle presence or absence information may be communicated as part of the dispenser data from the module 50 along with each dispense event and a count of the total number of dispense since bottle replacement.

Inclusion of a product bottle detection feature such as bottle presence trigger 21 allows tracking of the replacement of hand hygiene product in the dispenser, so the system can determine when product needs to be replaced and also that the product is replaced at the appropriate time. For example, a time/date stamped event may be recorded when a product bottle has been taken out of a dispenser (e.g., when the switch is opened) and another event may be recorded when a product bottle has been replaced into the dispenser (e.g., when the switch is closed). The module 50 or a remote computing system may count the number of dispenses since bottle replacement (e.g., a switch opening event followed by a subsequent switch closing event), and may count down the number of events to a predetermined “alert” level for replacement. The module 50 or a remote computing system may compare the number of dispense events that occurred at the time of bottle replacement to the predetermined alert level to determine whether the product bottle was replaced too early, thus possibly wasting hand hygiene product by incomplete emptying of the product bottle. Bottle presence trigger 21 also allows for the module 50 (or a remote computing system) and hand hygiene compliance personnel to identify when a dispenser is being used without any hand hygiene product (e.g., when actuation of the hand hygiene product dispenser is detected but the bottle presence switch is not closed). The module 50 or the remote computing system may generate an alert to communicate to hand hygiene compliance personnel that the hand hygiene product dispenser is being used without any hand hygiene product and to inform them that product needs to be installed in that particular dispenser.

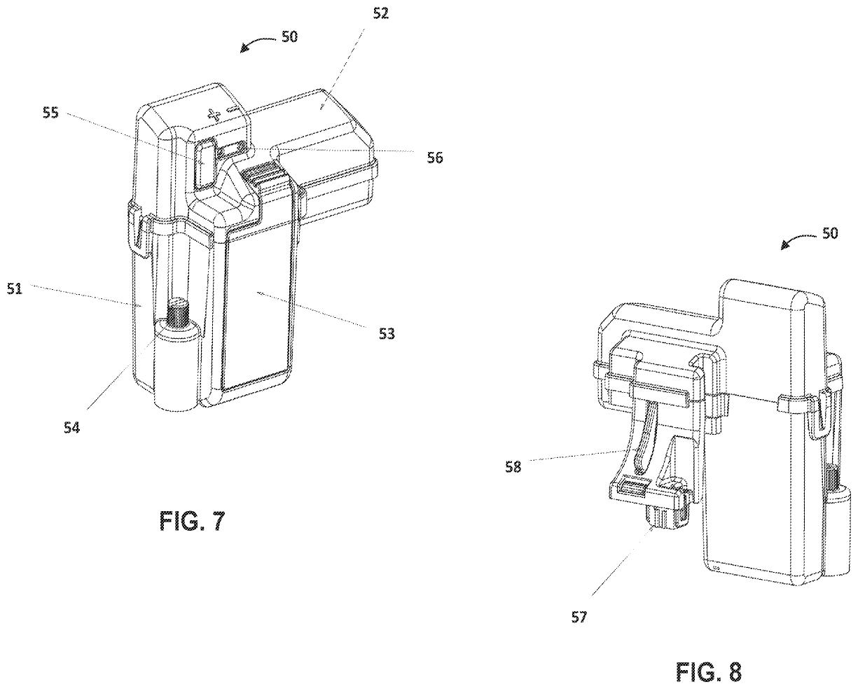

FIGS. 7 and 8 show a front perspective view and a back perspective view, respectively, of an example touch free dispenser beacon module 50. Touch free dispenser beacon module 50 is configured to be used with a touch free hand hygiene product dispenser to monitor hand hygiene events associated with the dispenser, and to wirelessly transmit hand hygiene data (including data concerning the monitored hand hygiene events) to or from the touch free dispenser 50. Touch free dispenser beacon module 50 includes a module base 51, a module cover 52, a battery door 53, a captive mounting screw 54, a firmware access port 35, a connector 57, and a bottle presence trigger 58 (implemented using a rocker arm assembly in this example).

In some examples, the touch free dispenser beacon module 50 is further configured to wirelessly transmit and/or receive communication from one or more computing device(s). For example, the beacon module 50 may receive remote software updates, remote configuration settings (e.g., range settings, product empty settings, settings for a number of dispense events before a product bottle should be refilled or replaced, etc.) from one or more computing devices. The beacon module 50 may further communicate with one or more other beacon modules in a healthcare setting, such as those associated with other dispensers, with motion detectors in a patient room or other defined area, with patient zone beacons in a patient room or other defined area, or other such devices in a healthcare setting that may be useful for monitoring of hand hygiene compliance. The beacon module 50 may be further configured to wirelessly communicate (both transmit and receive) with one or m ore uniquely assigned healthcare worker identification badges. For example, the beacon module 50 may be configured to communicate with a badge, obtain healthcare worker identification information from the badge, and associate a detected dispense event with the healthcare worker identification information,

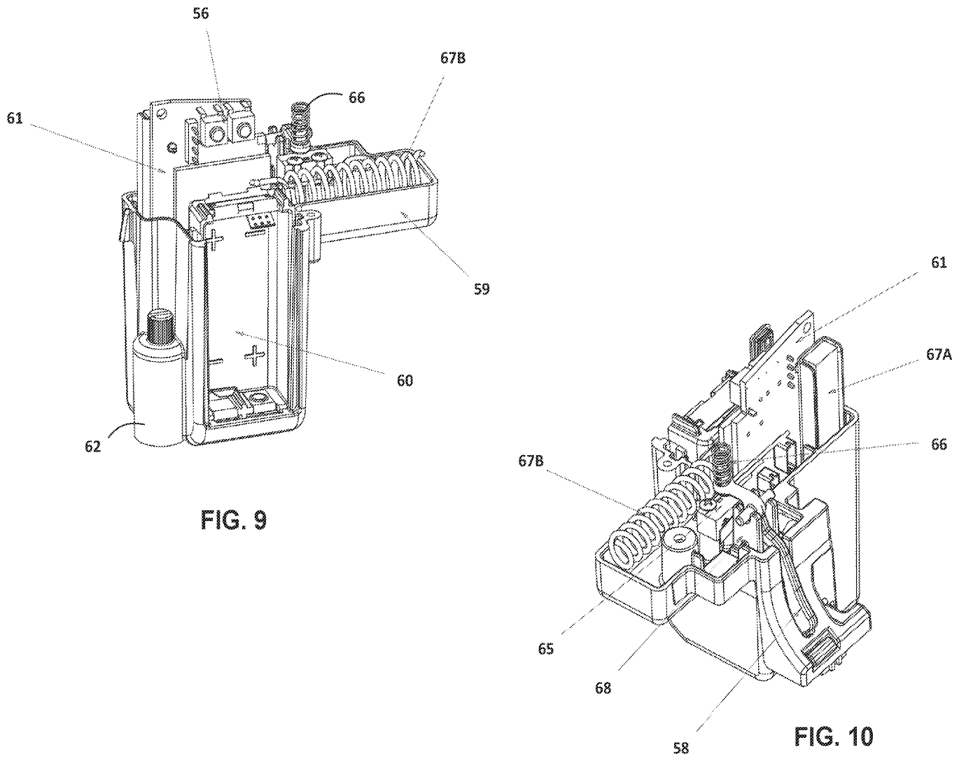

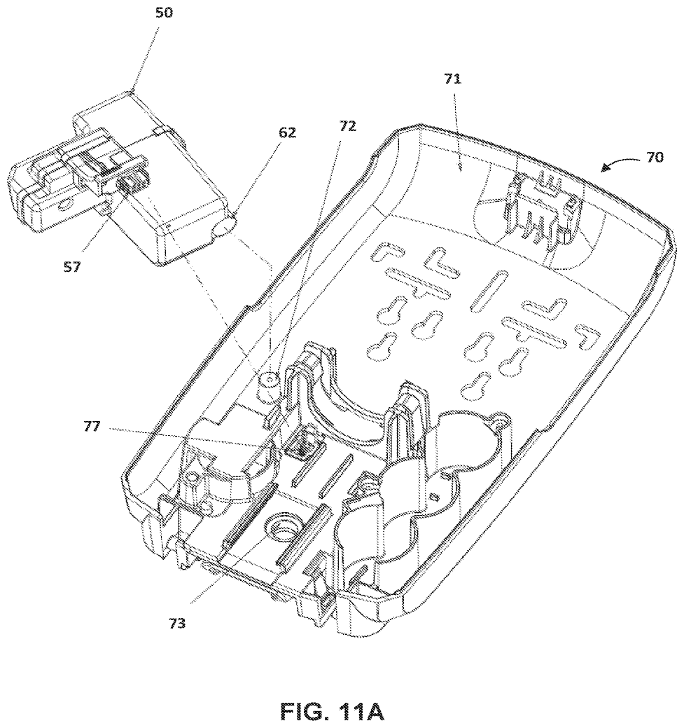

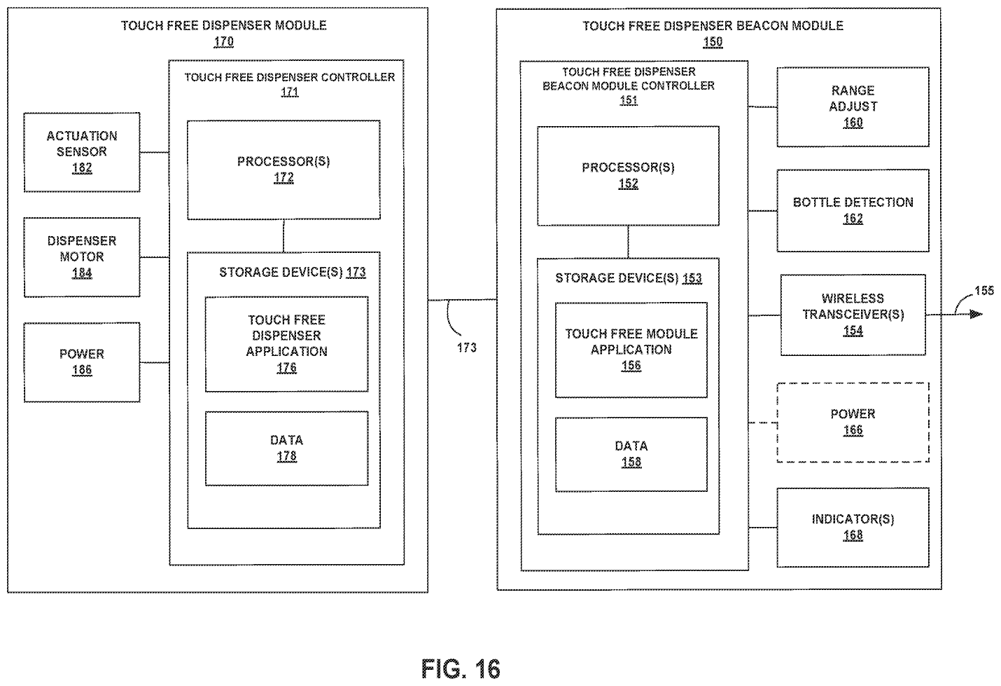

FIGS. 9 and 10 show a front perspective view and a back perspective view, respectively, of the internal components of example touch free dispenser beacon module 50 with module cover 52 removed. Touch free dispenser beacon module 50 includes a battery enclosure 60, a PCB assembly 61 including a controller (see FIG. 16), bottle presence trigger 58, bottle detection micro switch 65, rocker return spring 66, and connector/rocker retainer 68. PCT assembly 61 includes two antennas, a high frequency antenna 67A and a low frequency coil antenna 67B. Range buttons 56 are accessed through holes in module cover 52. In this example, range buttons 56 adjust the range of low frequency antenna 67A.

Battery enclosure 60 is connected to, and provides power to PCB assembly 61. In this example, touch free dispenser beacon module 50 is powered using 2 AA batteries. However, it shall be understood that other means of powering module 50 may be used, and that the disclosure is not limited in this respect. In other examples, manual dispenser beacon module may be powered using different types of batteries, may be hard-wired to the electrical system of the building, may receive power from the batteries or the controller of the touch free dispenser.

Dispenser/module communication connector 57 communicatively couples PCB assembly 61 (and thus the touch free dispenser beacon module controller) with the controller of a touch free dispenser.

Inclusion of a bottle detection feature such as bottle presence trigger 58 allows tracking of the replacement of hand hygiene product in the dispenser, so the beacon module 150 or a remote computing device or system can determine when hand hygiene product needs to be replaced. For example, a time/date stamped event may be recorded when a product bottle has been taken out and another time/date stamped event may be recorded when the product bottle has been replaced. The module or the system may count the number of dispenses since product bottle replacement, and may count down the number of events to a predetermined “alert” level for replacement. Bottle presence trigger 58 also allows for hand hygiene compliance personnel to identify when a dispenser is being used without any hand hygiene product, so that the likelihood of dispensers being used without any hand hygiene product is reduced. In general, some or all of the functionality described above with respect to the bottle detection feature of the manual hand hygiene product dispenser may also be implemented by the bottle detection feature of the touch free hand hygiene product dispenser.

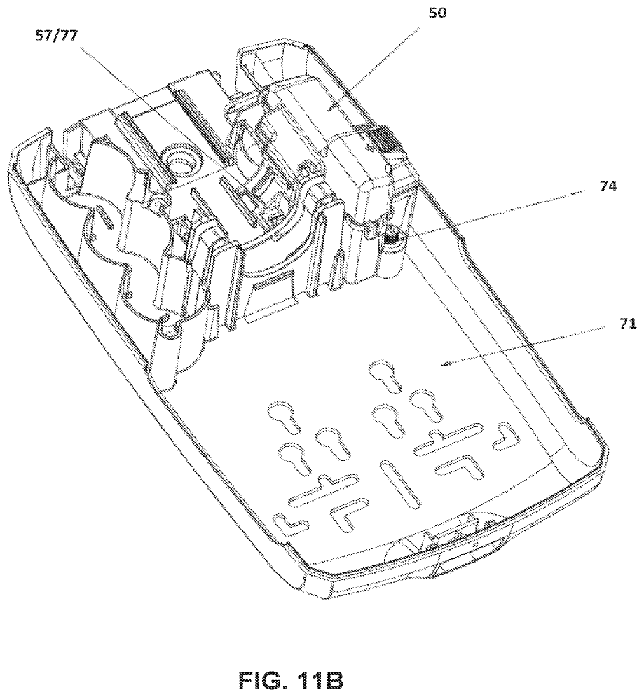

FIGS. 11A, 11B and 12-14 show various views of portions of an example touch free dispenser 70 and a touch free dispenser beacon module 50. Touch free dispenser 70 includes a base 71, a cover 73, and an electromechanical gearbox 73 that includes a communications connector 77. Communications connector 77 communicatively couples touch free dispenser controller (see FIG. 16) with the touch free dispenser beacon module controller (see FIG. 16). The touch free dispenser controller manages operation of ouch free dispenser 70, and includes a signal output indicative of actuation of the touch free dispenser, which is communicated to the touch free dispenser beacon module controller via the interface of connectors 57/77 as shown in FIG. 16. Base 71 of touch free dispenser 70 includes a mounting boss 72 that mates with a mounting receiver 62 on touch free module 50.

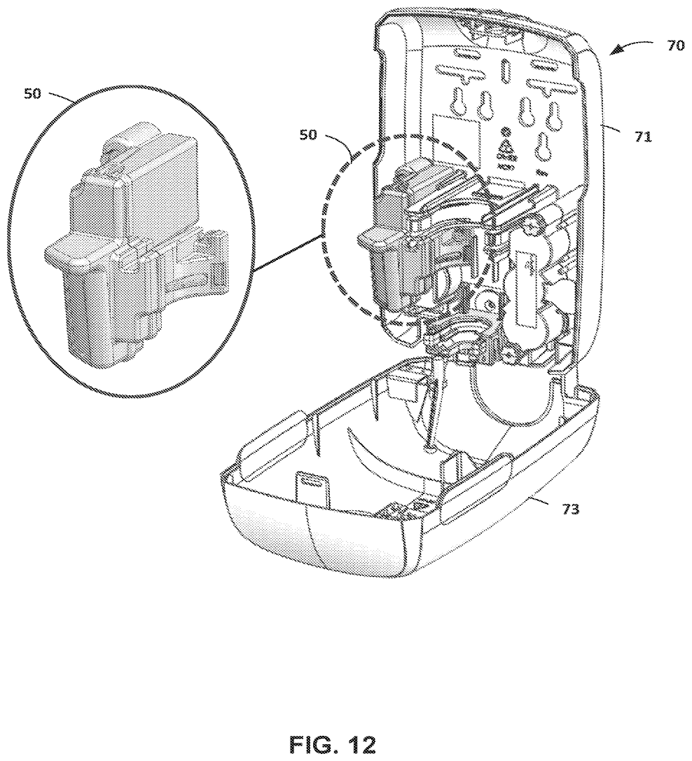

When touch free module 50 is installed into base 71 of touch free dispenser 70, as shown in FIGS. 11B and 12, connector 57 on touch free beacon module 50 is connected with connector 77 of the touch free dispenser controller, allowing dispenser controller and dispenser beacon module 50 to communicate. A mounting screw 74 may fit over the mating mounting boss 72 and fastens touch free module 50 to touch free dispenser 70.

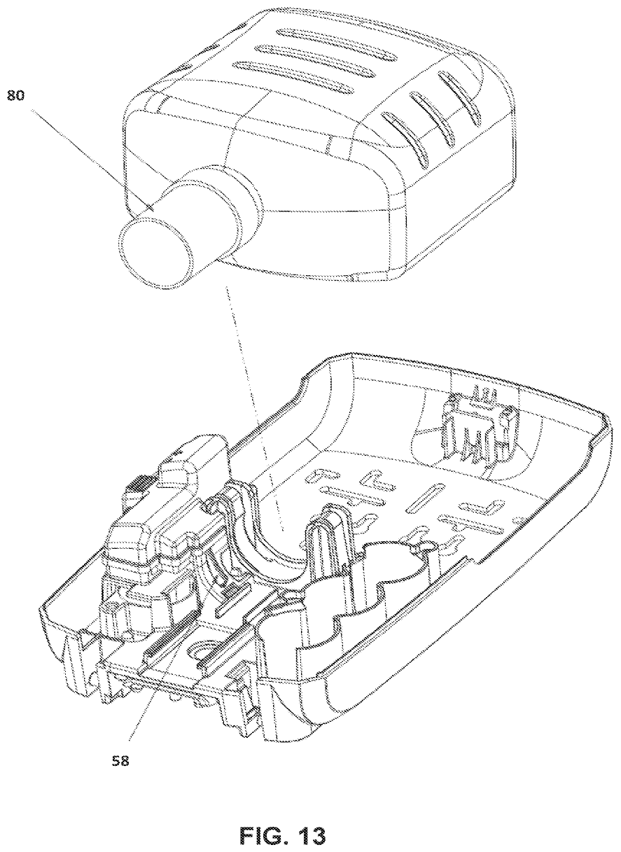

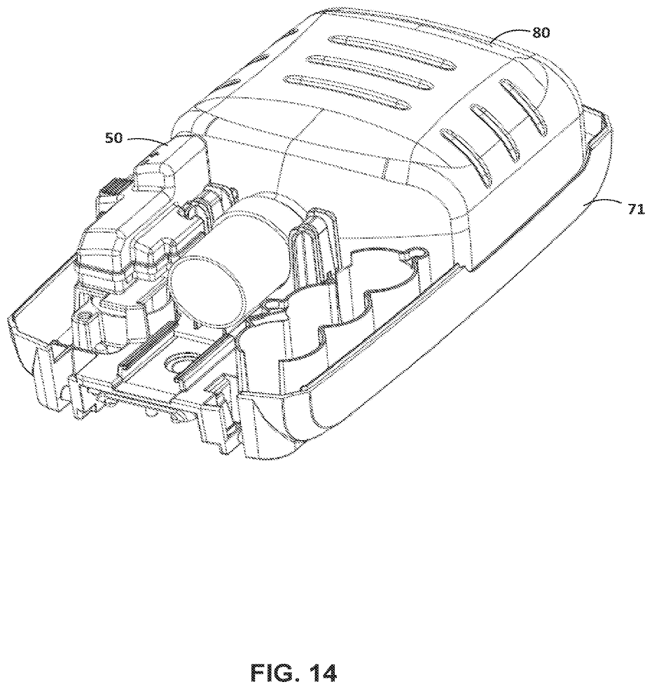

When there is no bottle installed in touch free dispenser 70, bottle detection rocker arm 58 is spring loaded by rocker return spring 66, compressing on the inside of module cover 52. When the system is at rest (i.e., no bottle installed in the dispenser), botte detection micro switch 65 is not pressed. When a bottle 30 is installed (FIGS. 13 and 14) the neck of the product bottle 80 presses and rotates rocker arm 58, depressing micro switch 65. Switch 65 communicates to the PCB assembly 61 that a bottle is installed. When the bottle 90 is removed, rocker arm 58 returns to its spring-loaded position releasing bottle detection micro switch 65, communicating to PCB assembly 61 that the bottle 80 has been removed.

FIG. 15 is a block diagram illustrating an example implementation of the electronic components of a manual dispenser beacon module 100. In this example, manual dispenser beacon module 100 includes a controller 101 that includes one or more processors 102 and storage device(s)/media 103. Processors 102, in one example, are configured to implement functionality and/or process instructions for execution within manual dispenser beacon module 100. For example, processors 102 may execute instructions stored in storage devices 103. Examples of processors 102 may include, any one or more of a microprocessor, a controller, a digital signal processor (DSP), an application specific integrated circuit (ASIC), a field-programmable gate array (FPGA), or equivalent discrete or integrated logic circuitry, including other hardware processors.

Example manual dispenser beacon module 100 further includes one or more wireless transceiver(s) 104, range adjustment buttons 110, a bottle detection switch 112, a manual dispenser actuation switch 114, a power supply 116, and one or more audible of visual indicators 118.

In some examples, the wireless transceiver(s) 104 of manual dispenser beacon module 100 is further configured to wirelessly transmit and/or receive communication from one or more computing device(s). For example, the beacon module 100 may receive remote software updates, remote configuration settings (e.g., range settings, product empty settings, settings for a number of dispense events before a product bottle should be refilled or replaced, etc.) from one or more computing devices. The beacon module 100 may further communicate with one or more other beacon modules in healthcare setting, such as those associated with other dispensers, with motion detectors in a patient room or other defined area, with patient zone beacons in a patient room or other defined area, or other such devices in a healthcare setting that may be useful for monitoring of hand hygiene compliance. The beacon module 100 may be further configured to wirelessly communicate (both transmit and receive) with one or more uniquely assigned healthcare worker identification badges. For example, the beacon module 100 may be configured to communicate with a badge, obtain healthcare worker identification information from the badge, and associate a detected dispense event with the healthcare worker identification information.

One or more storage devices 103 may be configured to store information within manual dispenser beacon module controller. Storage devices 103, in some examples, can be described as a computer-readable storage medium. In some examples, storage devices 103 are a temporary memory, meaning that a primary purpose of storage devices 103 is not long-term storage. Storage devices 103, in some examples, may be described as a volatile memory, meaning that storage devices 103 do not maintain stored contents when the computer is turned off. Examples of volatile memories include random access memories (RAM), dynamic random access memories (DRAM), static random access memories (SRAM) and other forms of volatile memories known in the art. In some examples, storage devices 103 are used to store program instructions for execution by processors 102, such as manual module application 106. Storage devices 103, in one example, are used by software or application 156 running on controller 101 to temporarily store information during program execution.

Storage devices 103, in some examples, also include one or more computer-readable storage media. Storage devices 103 may be configured to store larger amounts of information than volatile memory. Storage devices 103 may further be configured for long-term storage of information. In some examples, storage devices 103 may include non-volatile storage elements. Examples of such non-volatile storage elements include magnetic flash memories, or forms of electrically programmable memories (EPROM) or electrically erasable and programmable memories (EEPROM).

Storage device(s) 103 may store program instructions, such as touch free module application 156, for execution by processors 102. Manual module application 106 includes instructions that, when executed by processors 102, allow controller 101 to implement the manual dispenser beacon module functionality, such as monitor dispense events occurring, at the manual free dispenser. Store dispenser data concerning the dispense events, and wirelessly transmit (as indicated by reference numeral 105) the dispenser data via wireless transceiver 104. The dispenser data may include, for example, one or more of a dispenser id, a beacon module id, and a time and date stamp for each dispense event. The dispenser data may further include, for example, a current battery status, a total number of dispense events occurring during a predetermined time interval or since the last time the dispenser was refilled, a number of dispenses remaining before the dispenser runs out of hand hygiene product, an out-of-product or low product status, and/or other dispenser status information, etc.

Storage device(s) 103 may store various data (108) generated or used by processor(s) 102 during execution of the manual module application instructions 106. For example, storage device(s) may generate and store dispense event data, beacon module identification information, battery levels, bottle detection/presence information, range information, or other data associated with the manual dispenser beacon module 100.

Example manual dispenser beacon module 100 receives, for example, an indication of actuation of the manual dispenser from manual dispenser actuation switch 114. One example implementation for switch 114 is switch 19 of FIGS. 3 and 4. However it shall be understood that other implementations and mechanisms for detecting actuation of a manual dispenser may be used, and that the disclosure is not limited in this respect. Controller 101 may store information concerning the received indication as a dispense event in data storage 108. In some examples, controller 101 may attach a time and date stamp, dispenser identification information and/or beacon module identification information to the dispense event data. Controller 101 may wirelessly transmit (as indicated by reference numeral 105) via wireless transceiver(s) 104 the dispense event data upon receipt of each indication of a manual actuation, or may wirelessly transmit (as indicated by reference numeral 105) multiple dispense events on a periodic basis or on demand. In other examples, controller 101 wirelessly transmits (as indicated by reference numeral 105) via wireless transceiver(s) 104 dispenser data indicative of a dispense event upon receipt of each indication of dispenser actuation from manual dispenser actuation without appending a time and date stamp. A computing device configured to receive dispenser data from multiple manual and/or touch free dispenser beacon modules within a healthcare or other facility may associate each dispense event with a time and date stamp, and may analyze the dispense event data to monitor hand hygiene within the facility.

When beacon module 100 in installed in a manual hand hygiene product dispenser and a bottle is installed into the dispenser, bottle detection switch 112 such as switch 21 in FIGS. 1 and 5 is depressed (closed) and switch 112 generates a bottle present signal, which is in tur received by the beacon module controller 101. As long as a product bottle remains installed in the dispenser, the switch 112 remains closed and beacon module controller 101 may store the bottle present information in data store 108. If the bottle is removed, the switch 112 will return to the resting (open) state and the bottle present signal will no longer be present. Beacon module controller 101 may store information that no bottle is present in data store 108. Manual beacon module application 106 may cause processor(s) 103 to determine whether a bottle is present in the dispenser each time a dispense event occurs, and nay wirelessly transmit the bottle present information as part of the dispenser data each time a dispense event occurs. In this way, users may be informed as to whether a hand hygiene product is actually installed in the dispenser, and may take remedial measures (refill the dispenser with a product bottle) if the dispenser data indicates that no bottle is present.

FIG. 16 is a block diagram illustrating an example implementation of a touch free dispenser beacon module 150. In this example, touch free dispenser beacon module 150 further includes a controller 151 that includes one or more processors 152 and storage device(s)/media 153. Processors 152, in one example, are configured to implement functionality and/or process instructions for execution within touch free dispenser beacon module 150. For example, processors 152 may be capable of processing instructions stored in storage devices 153. Examples of processors 152 may include, any one or more of a microprocessor, a controller, a digital signal processor (DSP), an application specific integrated circuit (ASIC), a field-programable gate array (FPGA), or equivalent discrete or integrated logic circuitry, including other hardware processors.

Example touch free dispenser beacon module 150 further includes one or more wireless transceiver(s) 154, range adjustment buttons 160, a bottle detection switch 162, a power supply 166, and one or more audible of visual indicators 168.

In some examples, the wireless transceiver(s) 154 of touch free dispenser beacon module 150 is further configured to wirelessly transmit and/or receive communication from one or more computing device(s). For example, the beacon module 150 may receive remote software, updates, remote configuration settings (e.g., range settings, product empty settings, settings for a number of dispense events before a product bottle should be refilled or replaced, etc.) from one or more computing devices. The beacon module 150 may further communicate with one or more other beacon modules in healthcare setting, such as those associated with other dispensers, with motion detectors in a patient room or other defined area, with patient zone beacons in a patient room or other defined area, or other such devices in a healthcare setting that may be useful for monitoring of hand hygiene compliance. The beacon module 150 may be further configured to wirelessly communicate (both transmit and receive) with one or more uniquely assigned healthcare worker identification badges. For example, the beacon module 150 may be configured to communicate with a badge, obtain healthcare worker identification information from the badge, and associate a detected dispense event with the healthcare worker identification information.

One or more storage devices 153 may be configured to store information within touch free dispenser beacon module controller. Storage devices 153, in some examples, can be described as a computer-readable storage medium. In some examples, storage devices 153 are a temporary memory, meaning that a primary purpose of storage devices 153 is not long-term storage. Storage devices 153, in some examples, may be described as a volatile memory, meaning that storage devices 153 do not maintain stored contents when the computer is turned off. Examples of volatile memories include random access memories (RAM), dynamic random access memories (DRAM), static random access memories (SRAM), and other forms of volatile memories known in the art. In some examples, storage devices 153 are used to store program instructions for execution by processors 152, such as touch free module application 156. Storage devices 153, in one example, are used by software or application 156 running on controller 151 to temporarily store information during program execution.

Storage devices 153, in some examples, also include one or more computer-readable storage media. Storage devices 153 may be configured to store larger amounts of information than volatile memory. Storage devices 153 may further be configured for long-term storage of information. In some examples, storage devices 153 may include non-volatile storage elements. Examples of such non-volatile storage elements include magnetic flash memories, or forms of electrically programmable memories (EPROM) or electrically erasable and programmable memories (EEPROM).

Storage device(s) 153 may store program instructions, touch free module application 156, for execution by processors 152. Touch free module application 156 includes instructions that, when executed by processors 152, allow controller 151 to implement the touch free dispenser beacon module functionality, such as monitor dispense events occurring at the touch free dispenser, store dispenser data concerning the dispense events, and wirelessly transmit the dispenser data via wireless transceiver 154. The dispenser data may include, for example, one or more of a dispenser id, a beacon module id, and a time and date stamp for each dispense event. The dispenser data may further include, for example, a current battery status, a total number of dispense events occurring since a predetermined time interval or since the last time the dispenser was refiled, a number of dispenses remaining before the dispenser runs out of hand hygiene product, an out-of-product or low product status, and/or other dispenser status information, etc.

Storage device(s) 153 may store various data (158) generated or used by processor(s) 152 during execution of the touch free module application instructions 156. For example, storage device(s) may generate and store dispense event data, beacon module identification information, battery levels, bottle detection data, range information, or other data associated with the touch free dispenser beacon module 150.

Example touch free dispenser beacon module 150 electronically communicates with a touch free dispenser module 170 via communication link(s) 171. Touch free dispenser module 170 includes a touch free dispenser controller 171 that executes instructions stored on storage device(s) 173 to manage and control operation of a touch free dispenser, such as touch free dispenser 70. Dispenser controller 171 includes one or more processor(s) 172 and storage device(s) 173. A touch free dispenser application 176 stored in storage media 173 includes instructions that when executed by processors 172, implement control of the functionality for the touch free dispenser. Storage devices 173 may further include data 178 that is used or generated during execution of touch free dispenser application 176.

Touch fee dispenser module 170 further includes an actuation sensor 182 that senses actuation of the touch free dispenser and generates a corresponding actuation signal that is in turn received by controller 171. Actuation sensor 182 may include, for example, one or more of a photo interrupter, an infrared sensor, an optical sensor, a motion sensor, or other touchless or touch free mechanism for detecting presence of a user's hands. Touch free dispenser 170 further includes a dispenser motor 184 that is activated by controller 171 upon receipt for the actuation signal, thus causing a standardized dose of hand hygiene product to be dispensed from the touch free dispenser.

Communication link(s) 173 may be implemented in the example of FIGS. 11 and 12 via connectors 57/77. In this way, controller 151 of beacon module 150 receives, for example, an indication of touch free dispenser actuation from touch free dispenser controller 171 via communication link(s) 173. Controller 151 may store information concerning the received indication as a dispense event. In some examples, controller 151 may attach a time and date stamp, dispenser identification information and/or beacon module identification information to the dispense event data. Controller 151 may wirelessly transmit (as indicated by reference numeral 155) via wireless transceivers 154 the dispense event data upon receipt of each indication of a manual actuation, or may wirelessly transmit (as indicated by reference numeral 155) multiple dispense events on a periodic basis or on demand. In other examples, controller 151 wirelessly transmits (as indicated by reference numeral 155) via wireless transceivers 154 dispenser data indicative of a dispense event upon receipt of each indication of dispenser actuation from touch free dispenser controller without appending a time and date stamp. A computing device configured to receive dispenser data from multiple manual and/or touch free dispenser beacon modules within a healthcare or other facility may associate each dispense event with a time and date stamp, and may analyze the dispense event data to monitor hand hygiene within the facility.

Power source 166 is indicated in dashed lines to indicate that beacon module 150 power may alternatively by powered from touch free dispenser module 170. In such an example, instead of having dedicated batteries/power source 166, touch free dispenser beacon module 150 may be configured to receive power from the touch free dispenser 70. For example, controller 151 may receive power from touch free dispenser controller 170 via communication link(s) 173. This may reduce the overall physical size of the touch free dispenser beacon module 150, as it would not need to be sized to accommodate one or more batteries within the housing. The physical size and configuration of the housings for dispenser beacon module 50 shown in FIGS. 7-1 for example, may therefore be designed without a battery compartment 60 or battery cover 53, thus reducing the overall external dimensions of beacon module 50 and potentially making it easier to fit within the housing of a touch free dispenser.

In some examples there may be advantages to the touch free dispenser beacon module to have its own internal batteries. Each time the dispenser activates, a load is placed on the batteries. As the batteries approach the end of their life, their internal resistance increases and the load will cause the battery voltage to “droop” significantly, if the touch free dispenser beacon module is powered by the dispenser's batteries and if the battery voltage droops below the reset voltage threshold of the touch free beacon module controller, the touch free beacon module controller will be held in reset until the battery voltage recovers to a point above the reset threshold. Battery voltage recovery could take long enough to delay badge communication until the end of the dispense cycle. It could also take so long that the user which activated the dispenser has already left the area of the dispenser before the touch free beacon module controller has come out of reset and can communicate with that user's badge. The result may be that the user's badge has not been set to a clean hygienic state and the event is not reported. However, if the touch free beacon module has its own batteries, it is not affected by the voltage droop of the dispenser's batteries during activation and badge communication is more likely to ensue at the beginning of the dispense cycle.

Another benefit may be that a touch free beacon module, with its own batteries, will not reduce the life of the dispenser's batteries thus allowing the dispenser to meet specified battery life expectations. Also, the touch free beacon module may include the ability to monitor the level of the dispenser's batteries as well as its own batteries. It may be able to report the level of the dispenser's batteries even after their voltage has dropped below a level that would not allow the touch free beacon module to function had it been using the dispenser's batteries.

In some examples, a plurality of manual dispenser beacon module(s) 100 and/or touch free dispenser beacon module(s) 150 may be used to monitor hand hygiene compliance in a healthcare setting or other setting in which hand hygiene compliance monitoring is desired. For example, the modular hand hygiene compliance system may be adapted for use in applications such as hotel room cleaning, education facilities, long term care, restaurants, food service, food and beverage facilities, food packing, eating areas, rest rooms, food preparation areas, cooking areas, etc.

In such a system, each healthcare worker (HCW) is assigned a compliance badge that is uniquely associated with the HCW. Each time a HOW dispenses hand hygiene product from one of the manual or touch free dispensers having a manual beacon module 100 or touch free beacon module 150, the corresponding beacon module 100/150 may communicate with the HCW badge, receive HCW identification information from the badge, and associate the HCW identification information with the dispense event. Example dispenser data stored and/or wirelessly transmitted upon each dispenser actuation is shown in Table 1:

TABLE 1
Example Dispenser Data with HCW Badge ID

| | |
|---|---|
| Dispenser ID | 12345678 |
| Dispense event | Yes |
| Time and Date | 12:36:15, 6 MAR. 2015 |
| Badge ID | 9876543AB |
| Bottle presence | Yes |
| Battery level | 92% |
| Range setting | 2 |
| Dispense event count since | 78 |
| last product refill | |
| Dispenses remaining until | 547 |
| out of product/refill | |

In other examples, (such as those in which the beacon modules do not communicate with an id badge), the dispenser data may include only an indication of the dispense event and an indication of bottle presence (yes or no). In other examples, the dispenser data may include an indication of a valid battery voltage instead of or in addition to the current battery level. In other examples, the dispenser data may include any one or all of the example dispenser data listed in Table 1, and/or other dispenser data. The dispense event count since last product refill may be reset each time a product bottle removal/replacement is detected by bottle presence triggers of the manual or touch free beacon modules.

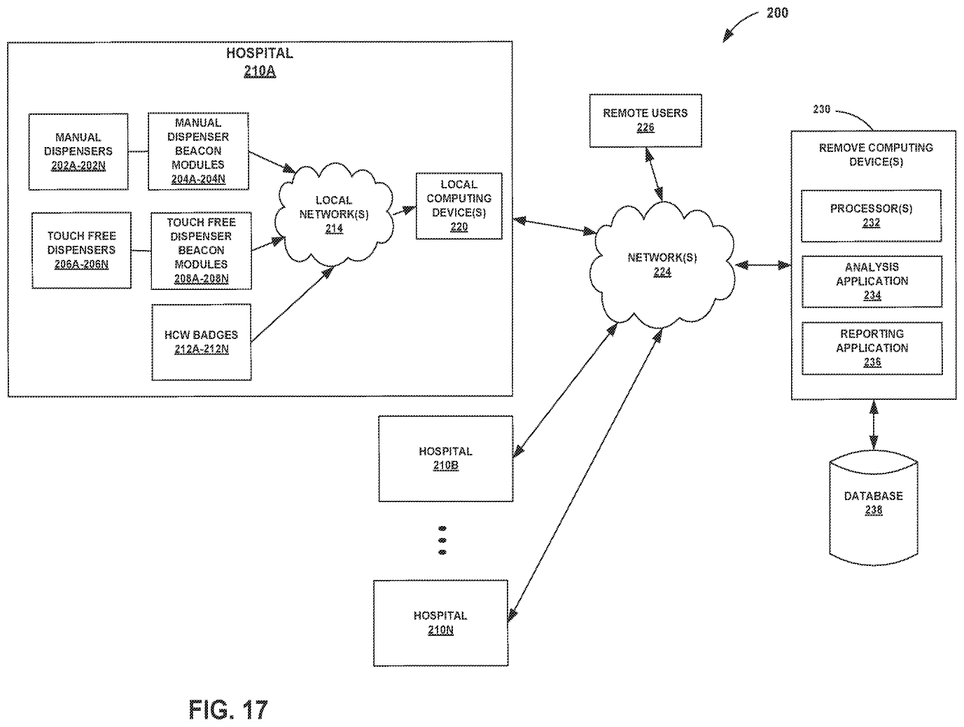

FIG. 17 is a block diagram of an example hand hygiene compliance monitoring system 200. A plurality of healthcare facilities, such as hospitals 210A-210N, each include a plurality of manual hand hygiene product dispensers 202A-202N and/or a plurality of touch free hand hygiene product dispensers 206A-206N. For simplicity of illustration, these are shown with respect to hospital 210A. Each of the plurality of manual dispensers 202A-202N is associated with a different one of a plurality of manual dispenser beacon modules 204A-204N that provide for wireless transmission of dispenser data. Similarly, each of the plurality of touch free dispensers 206A-206N is associated with a different one of a plurality of touch free dispenser beacon modules 208A-208N that provide for wireless transmission of dispenser data.

Dispenser beacon modules 203A-204N and 208A-208N wirelessly transmit their respective dispenser data to one or more local computing device(s) 220 via local network(s) 214. In the example where beacon modules transmit dispenser data upon the occurrence of each dispense event and does not include a time and date stamp in the dispenser data, local computing device will associate a time and date stamp with the dispense event.

In some examples, such as that shown in FIG. 17, hand-hygiene compliance monitoring system 200 includes HCW badges 212A-212N. In this example, therefore, the dispenser data transmitted by beacon modules 204A-204N and/or 208A-208N may include HCW identification information received from badges 212A-212N.

To monitor hand hygiene compliance, dispenser data from the plurality of dispenser beacon modules 100/150 are wirelessly transmitted to one or more local computing device(s) 220 located within the healthcare facility and/or to remote computing device(s) 230 for data analysis and reporting. As shown in FIG. 17, for example, computing devices 230 may include one or more processor(s) 232, an analysis application 234, a reporting application 236, and a data base 238 that stores the requisite data used or generated by system 200. Analysis application 234, when executed by processors 232, analyzes the hand hygiene data in accordance with one or more compliance rules so as to monitor hand hygiene compliance with the healthcare facility. Reporting application 2361 when executed by processors 232, generates reports regarding hand hygiene compliance. For example, computing devices 230 may analyze the hand hygiene data to monitor hand hygiene compliance by individual HCW, type of HCW (e.g., nurses, doctors, environmental services (EVS), etc.) department, type of department, individual hospital, type of hospital, across multiple hospitals, or by various other selected parameters. Computing devices 230 may generate a variety of reports to provide users local to each hospital 210A-210N or remote users 226 with both qualitative and quantitative data regarding hand hygiene compliance at their hospital, to compare data over time to determine whether improvement has occurred, and/or to benchmark hand hygiene compliance at one hospitals, at multiple hospitals, or to view and compare hand hygiene compliance over time. Analysis and reporting application may also be stored locally on hospital computing devices 220 so that analysis and reporting of hand hygiene data may be done locally if desired.

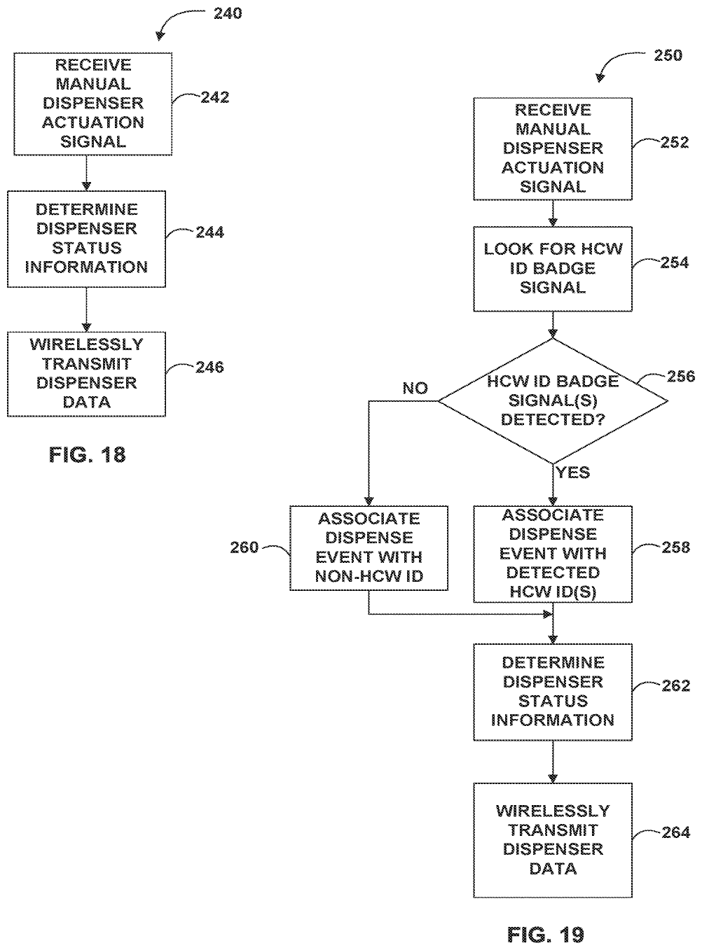

FIG. 18 is a flowchart illustrating an example process (240) by which a manual dispenser beacon module, such as beacon module 100, may detect manual actuations of a manual hand hygiene product dispenser and wirelessly transmit dispenser data associated with the dispense event. Beacon module 100 receives a dispenser actuation signal (242) indicative of actuation of the manual hand hygiene product dispenser. The actuation signal may be received from, for example, a switch configured to detect manual actuation of a manual hand hygiene product dispenser, such as switch 19 of FIGS. 3 and 4 and/or switch 114 of FIG. 15.

Beacon module 100 may further determine additional dispenser status information (244). For example, beacon module 100 may determine the current battery level, whether a bottle is present in the manual dispenser, may increment a count of the number of dispenses, may determine a number of dispenses remaining before the product bottle needs to be replaced or refilled, etc. Beacon module 100 then wirelessly transmits the dispense event data (246).

FIG. 19 is a flowchart illustrating another example process (250) by which a manual dispenser beacon module, such as beacon module 100, may detect manual actuations of a manual hand hygiene product dispenser and wirelessly transmit dispenser data associated with the dispense event. Beacon module 100 receives a dispenser actuation signal (252) indicative of actuation of the manual hand hygiene product dispenser. The actuation signal may be received from, for example, a switch configured to detect manual actuation of a manual hand hygiene product dispenser, such as switch 19 of FIGS. 3 and 4 and/or switch 114 of FIG. 15. In this example, beacon module controller 100 may then look for any HCW ID badge signals within range of the dispenser (254). For example, a wireless transceiver on beacon module controller may have an initial range of 0-1 meter or some other appropriate distance that helps to ensure that only the HCW ID badge associated with the HCW who initiated the dispense event is detected and not another nearby HCW id tag.

If a HCW ID badge signal is detected within a predefined period of time (256) (such as 0.5 seconds, 1 second, 2 seconds, 5 seconds or other appropriate time interval, for example), beacon module 100 associates the dispense event with the detected HCW identification information (258). If no HCW ID badge signal is detected within a predefined period of time, beacon module 100 associates the dispense event with non-HCW identification information (260).

Beacon module 100 may further determine additional dispenser status information (262). For example, beacon module 100 may determine the current battery level, whether a bottle is present in the manual dispenser, may increment a count of the number of dispenses, may determine a number of dispenses remaining before the product bottle needs to be replaced or refilled, etc. Beacon module 100 then wirelessly transmits the dispense event data (264).

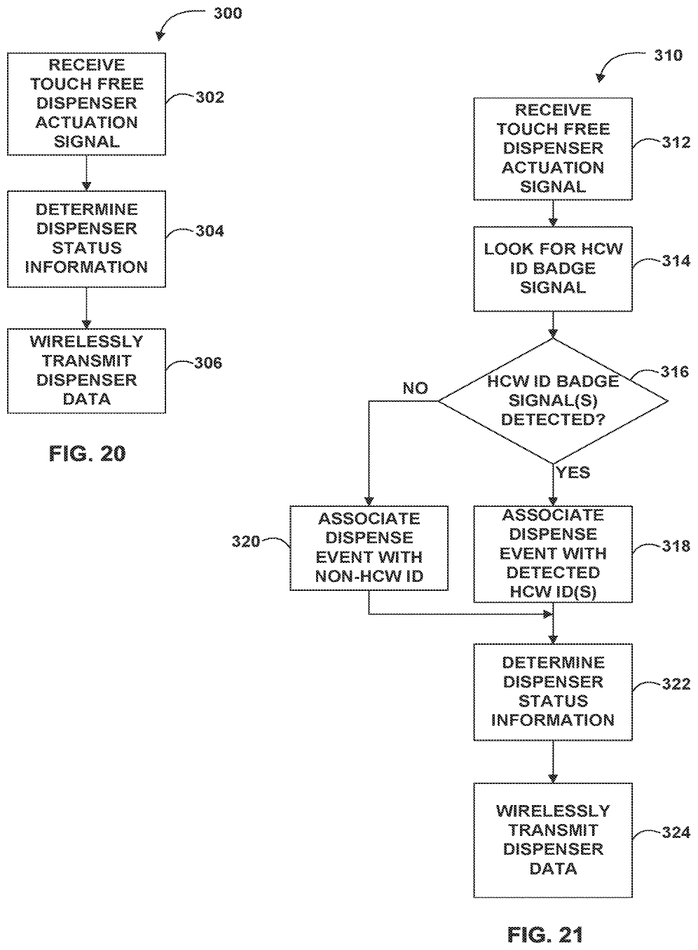

FIG. 20 is a flowchart illustrating an example process (300) by which a touch free dispenser beacon module, such as beacon module 150, may detect actuations of a touch free hand hygiene product dispenser and wirelessly transmit dispenser data associated with the dispense event. Beacon module 150 receives a dispenser actuation signal (302) indicative of actuation of the touch free hand hygiene product dispense. The actuation signal may be received from, for example, a touch free dispenser module (such as touch free dispenser module 170 of FIG. 16) that controls operation of, and thus detects actuation of, the touch free hand hygiene product dispenser.

Beacon module 150 may further determine additional dispenser status information (304). For example, beacon module 150 may determine the current battery level, whether a bottle is present in the touch free dispenser, may increment a count of the number of dispenses, may determine a number of dispenses remaining before the product bottle needs to be replaced or refilled, etc. Beacon module 150 then wirelessly transmits the dispense event data (306).

FIG. 21 is a flowchart illustrating another example process (310) by which a touch free dispenser beacon module, such as beacon module 150, may detect actuations of a touch free hand hygiene product dispenser and wirelessly transmit dispenser data associated with the dispense event. Beacon module 150 receives a dispenser actuation signal (312) indicative of actuation of the touch free hand hygiene product dispenser. The actuation signal may be received from, for example, a touch free dispenser module (such as touch free dispenser module 170 of FIG. 16) that controls operation of, and thus detects actuation of, the touch free hand hygiene product dispenser.

In this example, beacon module controller 150 may then look for any HCW ID badge signals within range of the dispenser (316). For example, a wireless transceiver on beacon module controller may have an initial range of 0-1 meter or some other appropriate distance that helps to ensure that only the HCW ID badge associated with the HCW who initiated the dispense event is detected and not another nearby HCW id tag.

If a HCW ID badge signal is detected within a predefined period of time (316) (such as 0.5 seconds, 1 second, 2 seconds, 5 seconds or other appropriate time interval, for example), beacon module 100 associates the dispense event with the detected HCW identification information (318). If no HCW ID badge signal is detected within a predefined period of time, beacon module 150 associates the dispense event with non-HCW identification information (320).

Beacon module 150 may further determine additional dispenser status information (322). For example, beacon module 150 may determine the current battery level, whether a bottle is present in the manual dispenser, may increment a count of the number of dispenses, may determine a number of dispenses remaining before the product bottle needs to be replaced or refilled, etc. Beacon module 150 then wirelessly transmits the dispense event data (324).

In accordance with one or more aspects of this disclosure, the term “or” may be interrupted as “and/or” where context does not dictate otherwise. Additionally, while phrases such as “one or more” or “at least one” or the like may have been used in some instances but not others, those instances where such language was not used may be interpreted to have such a meaning implied where context does not dictate otherwise.

In one or more examples, the functions described may be implemented in hardware, software, firmware, or any combination thereof. If implemented in software, the functions may be stored on or transmitted over, as one or more instructions or code, a computer-readable device or medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to non-transitory tangible computer-readable storage media. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storge devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Disk and disc, as used, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (SPs), general purpose microprocessors, application specific integrated circuits (ASICs), field programmable logic arrays (FPGA s), or other equivalent integrated or discrete logic circuitry. Accordingly, the term “processor,” as used may refer to any of the foregoing structure or any other structure suitable or implementation of the techniques described. In addition, in some aspects, the functionality described may be provided within dedicated hardware and/or software modules. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperating hardware writs, including one or more processors as described above, in conjunction with suitable software and/or firmware.

Various examples have been described. These and other examples are within the scope of the following claims.

1. (canceled)

2. A wireless device update system comprising:
a dispenser beacon module including:
a wireless transceiver configured to wirelessly communicate with a remote computing device;
a controller including one or more processors and one or more storage devices; and
wherein the dispenser beacon module is configured to receive remote software updates from the remote computing device via the wireless transceiver.

3. The system of claim 2, wherein the dispenser beacon module is configured to receive remote configuration settings from the remote computing device, the remote configuration settings including range settings.

4. The system of claim 2, wherein the dispenser beacon module is configured to receive remote configuration settings from the remote computing device, the remote configuration settings including product empty settings.

5. The system of claim 2, wherein the wireless transceiver is configured to wirelessly communicate with healthcare worker identification badges.

6. A dispenser beacon module configured to monitor actuations of a product dispenser, the dispenser beacon module comprising:
a controller including one or more processors and one or more storage devices;
a wireless transceiver configured to wirelessly communicate with a remote computing device; and
wherein the dispenser beacon module is configured to receive firmware update data from the remote computing device via the wireless transceiver.

7. The dispenser beacon module of claim 6, further comprising a module housing sized to fit within a housing of the product dispenser.

8. The dispenser beacon module of claim 6, wherein the dispenser beacon module is configured to receive remote configuration settings from the remote computing device, the remote configuration settings including range settings.

9. A dispenser beacon module comprising:
a controller including one or more processors and one or more storage devices;
a wireless transceiver configured to wirelessly communicate with a remote computing device and with healthcare worker identification badges; and
wherein the dispenser beacon module is configured to receive remote configuration settings from the remote computing device via the wireless transceiver.

10. The dispenser beacon module of claim 9, wherein the remote configuration settings include range settings for adjusting communication ranges of the wireless transceiver.

11. The dispenser beacon module of claim 9, wherein the remote configuration settings include product empty settings and settings for a number of dispense events before product replacement.

12. The dispenser beacon module of claim 9, further comprising range adjustment controls accessible from an exterior of the dispenser beacon module.

13. The dispenser beacon module of claim 9, wherein the wireless transceiver includes multiple antennas including a high frequency antenna and a low frequency coil antenna.

14. The dispenser beacon module of claim 9, wherein the controller is configured to store range configuration data and wirelessly transmit range setting information.

15. The dispenser beacon module of claim 9, wherein the controller is configured to communicate with uniquely assigned healthcare worker identification badges and associate detected dispense events with healthcare worker identification information.

16. A method comprising:
providing a dispenser beacon module having a wireless transceiver and a controller including one or more processors and one or more storage devices; and
receiving, at the dispenser beacon module, remote software updates from a remote computing device via the wireless transceiver.

17. The method of claim 16, further comprising receiving remote configuration settings including range settings from the remote computing device.

18. The method of claim 16, further comprising associating detected dispense events with healthcare worker identification information received from healthcare worker identification badges.

19. The method of claim 16, wherein the controller is configured to wirelessly communicate with one or more other beacon modules in a healthcare setting.

20. The method of claim 16, wherein the controller is configured to store range configuration data and wirelessly transmit range setting information.

21. The method of claim 20, further comprising associating detected dispense events with healthcare worker identification information received from healthcare worker identification badges.
